(19) United States
(12) Patent Application Publication
(10) Pub. No.: US 2016/0157731 A1
(43) Pub. Date: Jun. 9, 2016

(54) SYSTEM FOR PROTECTING MALE REPRODUCTIVE HEALTH

(71) Applicant: Intel Corporation, Santa Clara, CA (US)

(72) Inventors: MICHAEL MORAN, naas (IE); STEPHEN C. CHADWICK, Chandler, AZ (US); CHARLES BARON, Chandler, AZ (US); TOBIAS M. KOHLENBERG, Portland, OR (US)

(73) Assignee: Intel Corporation, Santa Clara, CA (US)

(21) Appl. No.: 14/560,337

(22) Filed: Dec. 4, 2014

(51) Int. Cl. A61B 5/01; A61B 5/00

(52) U.S. Cl. CPC A61B 5/01; A61B 5/746; A61B 5/0008; A61B 5/6823; A61B 5/4375; A61B 2560/0242

(57) ABSTRACT

The present application is directed to a system for protecting male reproductive health. An example system may comprise at least a temperature control portion and a monitoring device. The temperature control portion may include a thermal accumulation material to absorb heat generated by male genitalia. The monitoring device may include at least one sensor to sense an internal temperature corresponding to a surface temperature of the male genitalia, and possibly a second sensor to sense a temperature external to the temperature control portion. The monitoring device may compare the internal and/or external temperatures to threshold temperature levels, and may take action based on either/both temperatures being at or above the threshold temperature levels. For example, the monitoring device may further comprise a feedback module to generate feedback for alerting a user to a dangerous temperature condition and a communication module to send messages comprising the internal/external temperatures, alert messages, etc.

### TECHNICAL FIELD

The present disclosure relates to medical devices, and more particularly, to a system that may help protect male reproductive health at least through temperature monitoring and reporting.

### BACKGROUND

While modern technology has improved the overall quality of life for society in general, there may be some unintended consequences that accompany a modern lifestyle. For example, at least one negative condition that continues to plague men in today's society is infertility. There are many factors that may cause infertility to men, but one of the most obvious is excessive heat. The ideal temperature for sperm production is three to four degrees below normal human body temperature. The temperature of male genitalia (i.e., the testicles) becoming warmer may reduce sperm count. Obvious causes of overheating may include, but are not limited to, sauna, hot tubs, heating blankets, waterbeds, etc. Avoiding readily apparent sources of heat may help to maintain sperm count at a normal healthy level, which may increase the overall probability of conception. However, there may be instances where men may not be aware that damage is being done due to overheating. People are sitting for longer periods of time every day in various situations. They are riding bicycles, in cars and public transportation over greater distances, spending more time at work, relaxing at home in front of the television, and using computing devices in a variety of different settings. All of this sedentary activity may contribute to damaging temperature levels over long periods of time, let alone the damage that may be caused by placing warm electronic devices on a person's lap, which may unintentionally apply additional heat to a man's genitals.

Existing solutions for infertility mostly attempt to determine when a couple is most likely to be able to conceive, not with identifying and avoiding factors that may be causing infertility. For example, female body temperature, hormone levels in saliva, etc. may be measured to track the ovulation cycle (e.g., when conception may be most probable). Any preventative measures that may be directed to improving male fertility may comprise general advice given by a doctor in regard to recommended clothing, behavior, etc. While this course of action may lead to some improvement, it is far from being totally effective as it relies on the patient both knowing what situations are problematic regarding sperm production, and more importantly, being aware when such a problematic situation exists, which may not always be readily apparent even to a doctor.

### BRIEF DESCRIPTION OF THE DRAWINGS

Features and advantages of various embodiments of the claimed subject matter will become apparent as the following Detailed Description proceeds, and upon reference to the Drawings, wherein like numerals designate like parts, and in which:

FIG. 1 illustrates an example system for protecting male reproductive health in accordance with at least one embodiment of the present disclosure;

FIG. 2 illustrates an example configuration for a reporting device, monitoring device and sensor usable in accordance with at least one embodiment of the present disclosure;

FIG. 3 illustrates an example configuration for materials in a temperature control portion and an illustration of performance of the materials in accordance with at least one embodiment of the present disclosure; and

FIG. 4 illustrates example operations for protecting male reproductive health in accordance with at least one embodiment of the present disclosure.

Although the following Detailed Description will proceed with reference being made to illustrative embodiments, many alternatives, modifications and variations thereof will be apparent to those skilled in the art.

### DETAILED DESCRIPTION

The present application is directed to a system for protecting male reproductive health. An example system may comprise at least a temperature control portion and a monitoring device. The temperature control portion may include a thermal accumulation material to absorb heat generated by male genitalia. In at least one embodiment, an externally-situated heat reflective material may reflect external heat. The monitoring device may include at least one sensor to sense an internal temperature corresponding to a surface temperature of the male genitalia, and possibly a second sensor to sense a temperature external to the temperature control portion. The monitoring device may compare the internal and/or external temperatures to threshold temperature levels, and may take action based on either/both temperatures being at or above the threshold temperature levels. For example, the monitoring device may further comprise a feedback module to generate feedback for alerting a user to a dangerous temperature condition. The monitoring device may further comprise a communication module to send messages comprising the internal and/or external temperatures, alert messages, etc. In at least one embodiment, a reporting device may receive the messages from the monitoring device and may generate enhanced data by correlating the internal and/or external temperatures with context data. The enhanced data may be sent to a remote resource to, for example, analyze the temperature data based on the context data.

In at least one embodiment, a system for protecting male reproductive health may comprise, for example, a temperature control portion and a monitoring device. The temperature control portion may include at least a material to control a temperature of a system user's genitalia. The monitoring device may include at least a first sensor to sense an internal temperature, a feedback module to generate feedback to the system user and a communication module to transmit a message including at least the internal temperature.

In at least one embodiment, the material may be situated on an internal surface of the temperature control portion to direct heat away from the system user's genitalia. For example, the material may be a thermal accumulation material. The temperature control portion may further comprise a thermal reflective material situated on an external surface to reflect heat external to the temperature control portion. In at least one example implementation, a sensing portion of the first sensor may be integrated into the temperature control portion. The internal temperature may correspond to a sensed surface temperature of the system user's genitalia.

In the same or a different embodiment, the monitoring device may further comprise an operations module to control operations based at least on the internal temperature. For example, the operations module may further be to cause the communication module to transmit a message including at least the internal temperature. The operations module may further be to at least one of cause the feedback module to provide feedback to the user when it is determined that the internal temperature is at or above a threshold temperature level, or cause the communication module to transmit an internal temperature alert message when it is determined that the internal temperature is at or above a threshold temperature level. In at least embodiment, the monitoring device may further comprise a second sensor to sense a temperature external to the temperature control portion. The operations module may further be to cause the communication module to transmit a message including at least the external temperature. The operations module may further be to at least one of cause the feedback module to provide feedback to the user when it is determined that the external temperature data is at or above a threshold temperature level and cause the communication module to transmit an external temperature alert message when it is determined that the external temperature data is at or above a threshold temperature level. The feedback module may be to generate at least one of audible feedback, visible feedback or haptic feedback to the user.

Consistent with the present disclosure, an example device for reporting data regarding male reproductive health may comprise, for example, at least a communication module and a reproductive health module. The communication module may be to interact with at least one of a system for protecting male reproductive health or a remote resource. The reproductive health module may be to receive a message from the system for protecting male reproductive health via the communication module, the received message including at least one of an internal temperature corresponding to a surface temperature of a system user's genitalia or an external temperature corresponding to a temperature external to a temperature control portion of the system for protecting male reproductive health, generate enhanced data by correlating context data with at least one of the internal temperature or the external temperature and cause the communication module to transmit a message to the remote resource, the transmitted message including at least the enhanced data. Consistent with the present disclosure, an example method for protecting male reproductive health may comprise, for example, sensing an internal temperature corresponding to a surface temperature of male genitalia, determining whether the internal temperature is at or above a first threshold temperature level and causing feedback to be generated based on a determination that the internal temperature is at or above the first threshold temperature level.

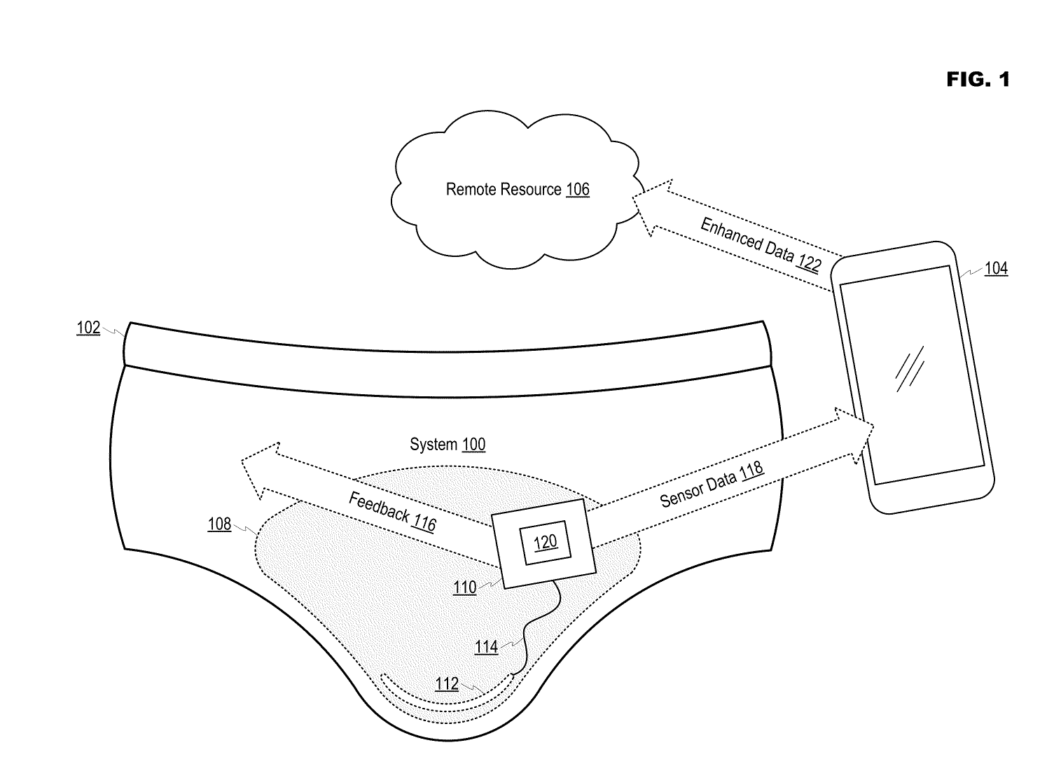

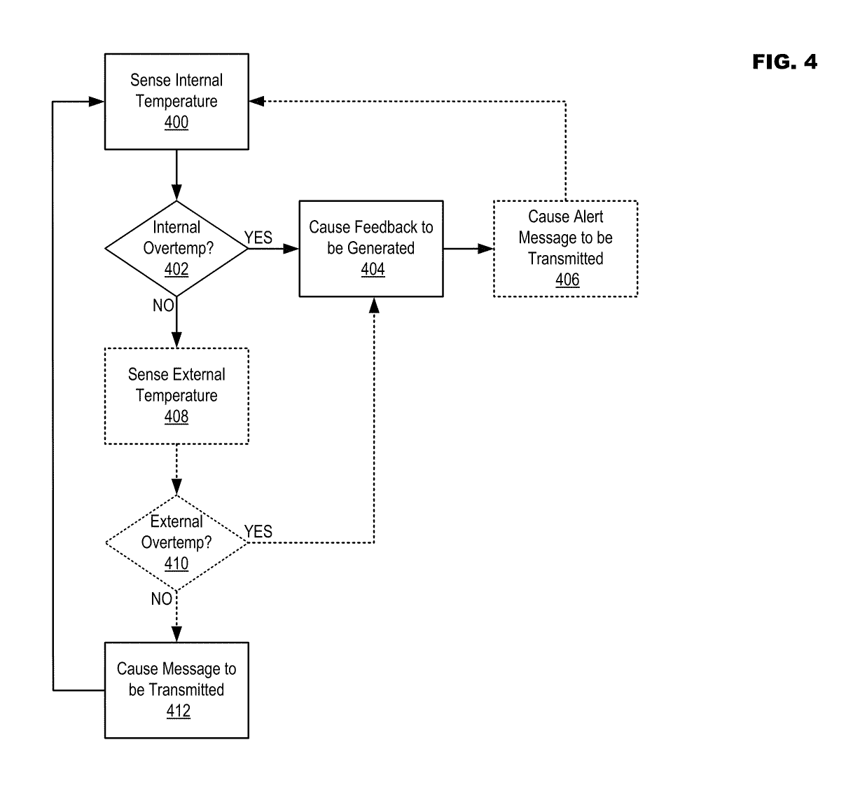

FIG. 1 illustrates an example system for protecting male reproductive health in accordance with at least one embodiment of the present disclosure. In general, system 100 may be affixed to, integrated within or simply placed within male undergarment 102 (e.g., underwear, underpants, briefs, boxer-type briefs, etc.), and may interact with at least one of reporting device 104′ and/or remote resource 106. System 100 may comprise, for example, at least temperature control portion 108 and monitoring device 110. Temperature control portion 108 may include a rigid, semi-rigid or pliable assembly formed to encompass at least the portion of the wearer's genitalia responsible for sperm production (e.g., the testicles) in a manner that allows for temperature control over the genitalia. For example, temperature control portion 108 may comprise a material situated at least on an interior surface so as to come into contact with the scrotum of a system user (e.g., a person that is wearing undergarment 102 including system 100). The material may direct heat away from the system user's genitalia to maintain genital temperature at or below a threshold temperature level for at least a period of time. The threshold temperature level may be, for example, a temperature above which human sperm production may be negatively impacted. In at least one embodiment, the material may be a thermal accumulation material functioning alone or in combination with a thermal reflective material in temperature control portion 108. Composition, behavior, etc. of materials that may be incorporated within temperature control portion 108 will be discussed in regard to FIG. 3.

Monitoring device 110 may be coupled to at least one sensor 112 (e.g., through wired or wireless connection 114), and may receive an internal temperature sensed by sensor 112. Sensor 112 may be a contact or non-contact temperature sensor capable of generating an analog and/or digital temperature. In at least one embodiment, at least a temperature sensing portion of temperature sensor 112 may be integrated into temperature control portion 108 (e.g., may be woven into, or placed between layers of, an interior material, affixed to a top surface of the interior material, etc.). The integration may be performed in a manner so that temperature sensor 112 may sense a surface temperature of the system user's genitalia, and is not substantially affected by (e.g., may be insulated from) the heat-reducing properties of temperature control portion 108. Sensing the surface temperature of the system user's genitalia without the influence of any heat-dissipative material in temperature control portion 108 may be important as at least one purpose of monitoring device 110 is to alert a user of system 100 of situations where sperm production may be compromised. It may therefore be more desirable to know of problematic heat-related situations while any heat control material within temperature control portion 108 may be still effective in maintaining genital temperature, not when the heat control material is already at or above the threshold temperature level. While monitoring device 110 has been illustrated as part of (e.g., integrated within or at least affixed to) temperature control portion 108, consistent with the present disclosure monitoring device 110 may also be situated apart from temperature control portion 108. For example, a monitoring device 110 separate from temperature control portion 108 may be affixed (e.g., clipped) to a waistband of undergarment 102.

Monitoring device 110 may receive at least the internal temperature sensed by sensor 112 (e.g., via wired or wireless connection 114), and may perform a variety of activities based on the internal temperature. In at least one embodiment, monitoring device 110 may generate feedback 116 to the system user. Feedback 116 may include at least one of audible feedback (e.g., sound such as a tone, bell, buzzer, etc.), visual feedback (e.g., an indicator that lights up, blinks, etc.) or haptic feedback (e.g., a vibration that may be felt by the system user). Of all of these options for feedback 116, haptic feedback may be preferable to preserve the privacy of the system user. It may also be possible for monitoring device 110 to transmit sensor data 118 to reporting device 104. For example, monitoring device 110 may comprise resources for formulating a message for transmission via wireless communication, the message including at least the internal temperature sensed by sensor 112. In at least one embodiment, monitoring device 110 may further comprise a sensor 120 to sense a temperature external to temperature control portion 108 (e.g., an environmental temperature). While external temperature sensor 120 has been represented as integrated into, or at least coupled to, monitoring device 110, it may also be possible for sensor 120 to be situated apart from monitoring device 110 such as described with respect to internal temperature sensor 112. Monitoring device 110 may cause the external temperature to be transmitted to reporting device 104 in the same message as the internal temperature or in a separate message. It may be important to sense the external temperature to identify situations that may cause an unwanted increase in genital temperature to or above the threshold temperature level. This may allow for the generation of feedback 116 prior to the occurrence of overheating that may negatively impact sperm production, for a third party (e.g., a doctor, nurse practitioner, nurse, technician, etc.) to identify impending overheating situations and/or to warn the system user of such situations, etc.

In general, reporting device 104 may be any device capable of receiving sensor data 118 from monitoring device 110 and reporting enhanced data 122. Examples of reporting device 104 may comprise, but are not limited to, mobile communication devices such as a cellular handset, smart phone, etc. based on the Android® operating system (OS) from the Google Corporation, iOS® from the Apple Corporation, Windows® OS from the Microsoft Corporation, Mac OS from the Apple Corporation, Tizen™ OS from the Linux Foundation, Firefox® OS from the Mozilla Project, Blackberry® OS from the Blackberry Corporation, Palm® OS from the Hewlett-Packard Corporation, Symbian® OS from the Symbian Foundation, etc., mobile computing devices such as a tablet computer like an iPad® from the Apple Corporation, Surface® from the Microsoft Corporation, Galaxy Tab® from the Samsung Corporation, Kindle Fire® from the Amazon Corporation, etc., an Ultrabook® including a low-power chipset manufactured by Intel Corporation, netbooks, notebooks, laptops, palmtops, etc., wearable devices such as wristwatch form factor computing devices like the Galaxy Gear® from the Samsung Corporation, the Apple Watch from the Apple Corporation, the Moto 360 from the Motorola Corporation, etc., eyewear form factor interfaces like Google Glass® from the Google Corporation, etc.

Consistent with the present disclosure, reporting device 104 may receive sensor data 118 via wired communication, close-proximity wireless communication, short-range wireless communication, etc. Enhanced data 122 may then be generated based on determining a context for sensor data 118 and then correlating the context data with the temperature data. For example, sensor data 118 may be transmitted to reporting device 104 along with temporal data (e.g., time, date, etc.) corresponding to when sensor data 118 was sensed. Reporting device may then utilize internal resources (e.g., scheduling data, calendar data, positioning data, application data, etc.) and/or external resources (e.g., Internet-based resources such as mapping websites, social media, etc.) to determine contexts corresponding to internal and/or external temperatures in sensor data 118. The determined contexts may be correlated to sensor data 118 (e.g., combined with sensor data 118 in a manner to describe a context for each internal and/or external temperature) to result in enhanced data 122. In an example of operation, sensor data 118 may identify times on certain days during which genital overheating may occur. The time periods of genital overheating may then be correlated (e.g., based on date and time) to various locations including, for example, work, home, driving or commuting, gym, spa, etc. (e.g., utilizing locational data such as global positioning system (GPS) coordinates, wireless signals sensed from at least one nearby network access point (AP), motion sensing, acceleration sensing, etc.), to various activities including, for example, working, commuting, exercising, watching television, sleeping, etc. (e.g., utilizing device orientation sensing, application data, Internet data such as map location identification data, scheduling data, interactional data such as emails, messaging, social media updates, etc.). Thus, a period of overheating may be tied to riding a bicycle or sitting in a car for an extended period of time, working out at a gym, sitting in a hot tub, etc. In at least one embodiment, at least one application related to male reproductive health in reporting device 104 may utilize enhanced data 122 to, for example, present overheating analysis, generate warnings, provide advice, etc. Enhanced data 122 may further be transmitted to remote resource 106.

In at least one embodiment, remote resource 106 may comprise, for example, at least one computing device accessible to reporting device 104 via a network such as a local area-network (LAN), a wide-area network (WAN) like the Internet, a global-area network (GAN), etc. In at least one example implementation, remote resource 106 may comprise at least one data server operating in a cloud computing architecture. Remote resource 106 may receive enhanced data 122 from at least one reporting device 104, and may be accessible to system users, third parties working with the system users, etc. for monitoring male reproductive health. Monitoring male reproductive health may comprise, for example, reviewing enhanced data 122 and/or analysis data based on enhanced data 122, etc., and utilizing this information to alert the system user to problematic situations, discuss behavioral changes to avoid problematic situations, etc. It may also be possible for remote resource 106 to accumulate data from a plurality of reporting devices 104 for use in research (e.g., determining trends via statistical analysis, testing hypotheses, etc.). Thus, the data in remote resource 106 may not only be made available to third parties working with a system user (e.g., in a doctor/patient scenario), but also to researchers. In such instances, the source of enhanced data 122 may be anonymized to protect the privacy of the system user.

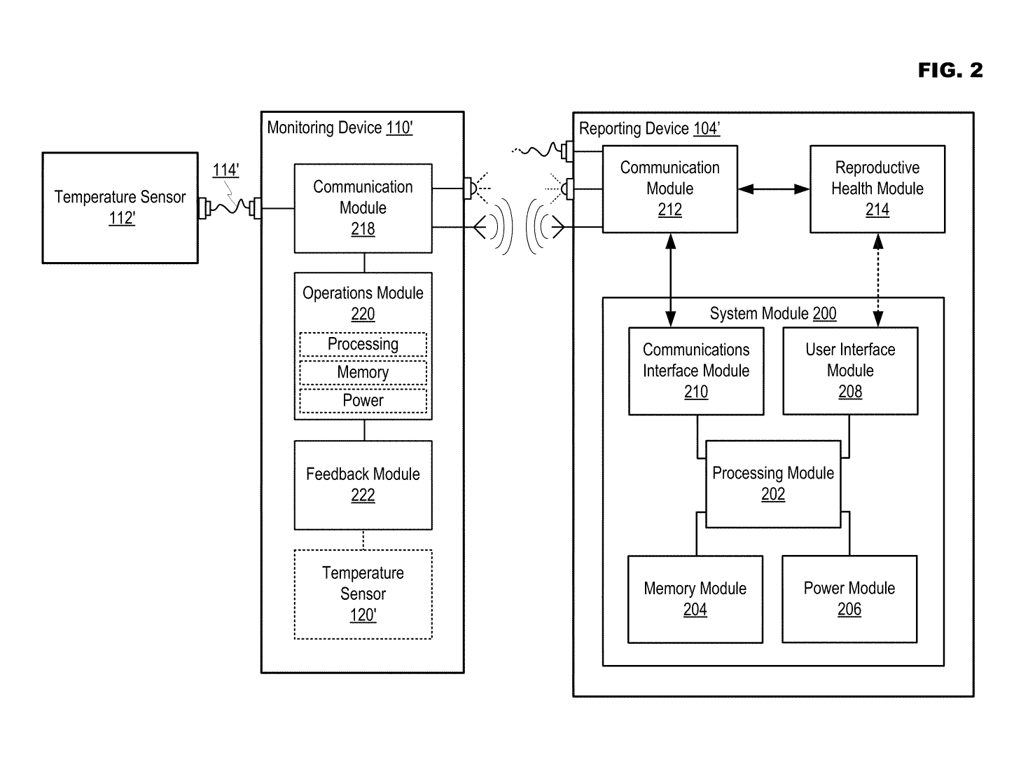

FIG. 2 illustrates an example configuration for a reporting device, monitoring device and sensor usable in accordance with at least one embodiment of the present disclosure. Reporting device 104′, monitoring device 110′ and/or temperature sensor 112′ may be capable of executing example functionality such as disclosed in FIG. 1. However, the devices illustrated in FIG. 2 are meant only as examples of devices usable in embodiments consistent with the present disclosure, and are not meant to limit the various embodiments to any particular manner of implementation.

Reporting device 104′ may comprise, for example, system module 200 configured to manage device operations. System module 200 may include processing module 202, memory module 204, power module 206, user interface module 208 and communication interface module 210. Reporting device 104′ may further include communication module 212 and reproductive health module 214. While communication module 212 and reproductive health module 214 have been shown as separate from system module 200, the example implementation illustrated in FIG. 2 has been provided merely for the sake of explanation. Some or all of the functionality associated with communication module 212 and/or reproductive health module 214 may be incorporated into system module 200.

In reporting device 104′, processing module 202 may comprise one or more processors situated in separate components, or alternatively, one or more processing cores embodied in a single component (e.g., in a System-on-a-Chip (SoC) configuration) and any processor-related support circuitry (e.g., bridging interfaces, etc.). Example processors may include, but are not limited to, various x86-based microprocessors available from the Intel Corporation including those in the Pentium, Xeon, Itanium, Celeron, Atom, Core i-series, Quark product families, Advanced RISC (e.g., Reduced Instruction Set Computing) Machine or “ARM” processors, etc. Examples of support circuitry may include chipsets (e.g., Northbridge, Southbridge, etc. available from the Intel Corporation) configured to provide an interface through which processing module 202 may interact with other system components that may be operating at different speeds, on different buses, etc. in reporting device 104′. Some or all of the functionality commonly associated with the support circuitry may also be included in the same physical package as the processor (e.g., such as in the Sandy Bridge family of processors available from the Intel Corporation).

Processing module 202 may be configured to execute various instructions in reporting device 104′. Instructions may include program code configured to cause processing module 202 to perform activities related to reading data, writing data, processing data, formulating data, converting data, transforming data, etc. Information (e.g., instructions, data, etc.) may be stored in memory module 204. Memory module 204 may comprise random access memory (RAM) or read-only memory (ROM) in a fixed or removable format. RAM may include volatile memory configured to hold information during the operation of reporting device 104′ such as, for example, static RAM (SRAM) or Dynamic RAM (DRAM). ROM may include non-volatile (NV) memory modules configured based on BIOS, UEFI, etc. to provide instructions when reporting device 104′ is activated, programmable memories such as electronic programmable ROMs (EPROMS), Flash, etc. Other fixed/removable memory may include, but are not limited to, magnetic memories such as, for example, floppy disks, hard drives, etc., electronic memories such as solid state flash memory (e.g., embedded multimedia card (eMMC), etc.), removable memory cards or sticks (e.g., micro storage device (uSD), USB, etc.), optical memories such as compact disc-based ROM (CD-ROM), Digital Video Disks (DVD), Blu-Ray Disks, etc.

Power module 206 may include internal power sources (e.g., a battery, fuel cell, etc.) and/or external power sources (e.g., power grid, electromechanical or solar generator, external fuel cell, etc.), and related circuitry configured to supply reporting device 104′ with the power needed to operate. User interface module 208 may include hardware and/or software to allow users to interact with reporting device 104′ such as, for example, various input mechanisms (e.g., microphones, switches, buttons, knobs, keyboards, speakers, touch-sensitive surfaces, one or more sensors configured to capture images and/or sense proximity, distance, motion, gestures, orientation, biometric data, etc.) and various output mechanisms (e.g., speakers, displays, lighted/flashing indicators, electromechanical components for vibration, motion, etc.). Any hardware needed to support functionality in user interface module 208 may be incorporated within reporting device 104′ and/or may be coupled to reporting device 104′ via a wired or wireless communication medium.

Communication interface module 210 may be configured to manage packet routing and other control functions for communication module 212, which may include resources configured to support wired and/or wireless communications. In some instances, reporting device 104′ may comprise more than one communication module 212 (e.g., including separate physical interface modules for wired protocols and/or wireless radios) managed by a centralized communication interface module 210. Example wired communications may include serial and parallel wired mediums such as Ethernet, Universal Serial Bus (USB), Firewire, Thunderbolt, Digital Video Interface (DVI), High-Definition Multimedia Interface (HDMI), etc. Example wireless communications may include close-proximity wireless mediums (e.g., radio frequency (RF) communications such as based on the RF identification (RFID) or Near Field Communications (NFC) standards, infrared (IR), body-coupled communications, etc.), short-range wireless mediums (e.g., Bluetooth, WLAN, Wi-Fi, etc.), long range wireless mediums (e.g., cellular wide-area radio communication technology, satellite-based communications, etc.), electronic communications via sound waves, etc. In one embodiment, communication interface module 210 may be configured to prevent wireless communications that are active in communication module 212 from interfering with each other. In performing this function, communication interface module 210 may schedule activities for communication module 212 based on, for example, the relative priority of messages awaiting transmission. While the embodiment disclosed in FIG. 2 illustrates communication interface module 210 being separate from communication module 212, it may also be possible for the functionality of communication interface module 210 and communication module 212 to be incorporated into the same module.

Consistent with the present disclosure, reproductive health module 214 may interact with at least communication module 212 and optionally with user interface module 208. For example, reproductive health module may receive at least sensor data 118 from monitoring device 110′ via communication module 212, and may transmit at least enhanced data 122 to remote resource 106 via communication module 212. Reproductive health module 214 may further interact with user interface module 208 to, for example, present a configuration interface allowing the system user to configure at least reproductive health module 214 (e.g., to configure the communication link between monitoring device 110′ and/or remote resource 106, the transmission rate of enhanced data 122, what context data may be considered in generating enhanced data 122, privacy settings in regard to enhanced data 122, etc.). Moreover, reproductive health module 214 may cause user interface module 208 be able to present raw data (e.g., sensor data 118) or processed data (e.g., showing trends regarding genital overheating, problematic scenarios regarding time of day, location, etc.) to present an interface for corresponding with a third party (e.g., a medical practitioner), etc.

Monitoring device 110′ may comprise, for example, at least communication module 218, operations module 220 and feedback module 222. Operations module 220 may comprise at least processing resources, memory resources and power resources in a single module. For example, operations module 220 may comprise processing/memory/power resources for receiving internal temperatures sensed by temperature sensor 112′, causing communication module 218 to transmit the internal temperatures to reporting device 104′ (e.g., utilizing close-proximity or short range wireless communication) and controlling feedback module 222. Feedback module 222 may comprise electronic and/or electromechanical equipment for generating at least one of audible feedback, visible feedback or haptic feedback to the user (e.g., due to an overheating situation). In at least one embodiment, monitoring device 110′ may further comprise, or may at least be coupled to, temperature sensor 120′ configured to sense an external temperature. Operations module 220 may further cause communication module 218 to transmit the external temperature to reporting device 104′ in a separate message or in combination with the internal temperature. In one example implementation, communication module 218, operations module 220 and/or temperature sensor 120′ may be consolidated into a single SoC, a multichip module (MCM), etc. While temperature sensor 112′ is shown coupled to communication module 218′ via wired connection 114′, this connection may also be made utilizing wireless communication.

FIG. 3 illustrates an example configuration for materials in a temperature control portion and an illustration of performance of the materials in accordance with at least one embodiment of the present disclosure. A cross-section diagram comprising example materials that may make up temperature control portion 108 is disclosed at 108′ in FIG. 3. In at least one embodiment, temperature control portion 108′ may include at least a material to control a temperature of a system user's genitalia as shown at 300. Material 300 may direct heat generated by the system user's genitalia away to maintain a genital temperature at or below the threshold level. Example materials 300 that may be able to perform this function may comprise, but are not limited to, heat conductive materials and thermal accumulation materials. Heat conductive or “sink” materials are generally known to be effective for cooling (e.g., a heat sink for a microprocessor), but may be of limited use when a system user is fully clothed since there may not be a way to dissipate the heat. However, thermal accumulation materials do not have this problem since they absorb heat. An example thermal accumulation material for textile from Venture Chemical Limited may be composed of phase-change material (PCM) and micro-capsule-material. An example PCM material may comprise paraffin capsules in a solid state. The capsules may store heat as they turn from a solid phase to a liquid phase, and may later release the heat when the capsules return back to the solid phase. This process may occur repeatedly as temperature rises and again falls, repeated absorbing heat from the system user's genital area, which may help to maintain genital temperature. Chart 304 illustrates example temperature behavior for a conventional material vs. a thermal accumulation material. As a temperature of a heat source (e.g., system user's genitalia) rises, temperature 306 of the conventional material crosses target ceiling temperature 308 at time 310 and continues to rise until it reaches the heat source actual temperature 312 at time 314. During the time period between times 310 and 314, the temperature of the conventional material is above target ceiling temperature 308 (e.g., which may reduce sperm production). However, as illustrated at 316, the thermal accumulation material is able to absorb heat generated by the heat source at least during the time period between times 310 and 314. This heat absorption may cause a delay 318 before the thermal accumulation material experiences any substantial temperature increase at time 314. Delay 318 may allow the system user to recognize (e.g., be alerted to) an overheating situation, and to remedy the overheating situation, before the negative impact of the overheating occurs.

In at least one embodiment, temperature control portion 108′ may also comprise heat reflective material 302 on an external surface. Heat reflective material 302 may reflect environmentally-produced heat away from temperature control portion 108′, and thus, may reduce the impact of this heat. In an example implementation including both external temperature sensor 120 and heat reflective material 302, external temperature sensor 120 may be situated outside of heat reflective material 302 so that the sensed external temperature may not be reduced by material 302′s heat reflective properties. In this manner, system 100 may be alerted to a problematic environmental scenario (e.g., sitting too long, a hot car seat, a hot tub, a sauna, etc.) before an internal temperature rise occurs due to the external heat, allowing the system user to immediately remedy the situation.

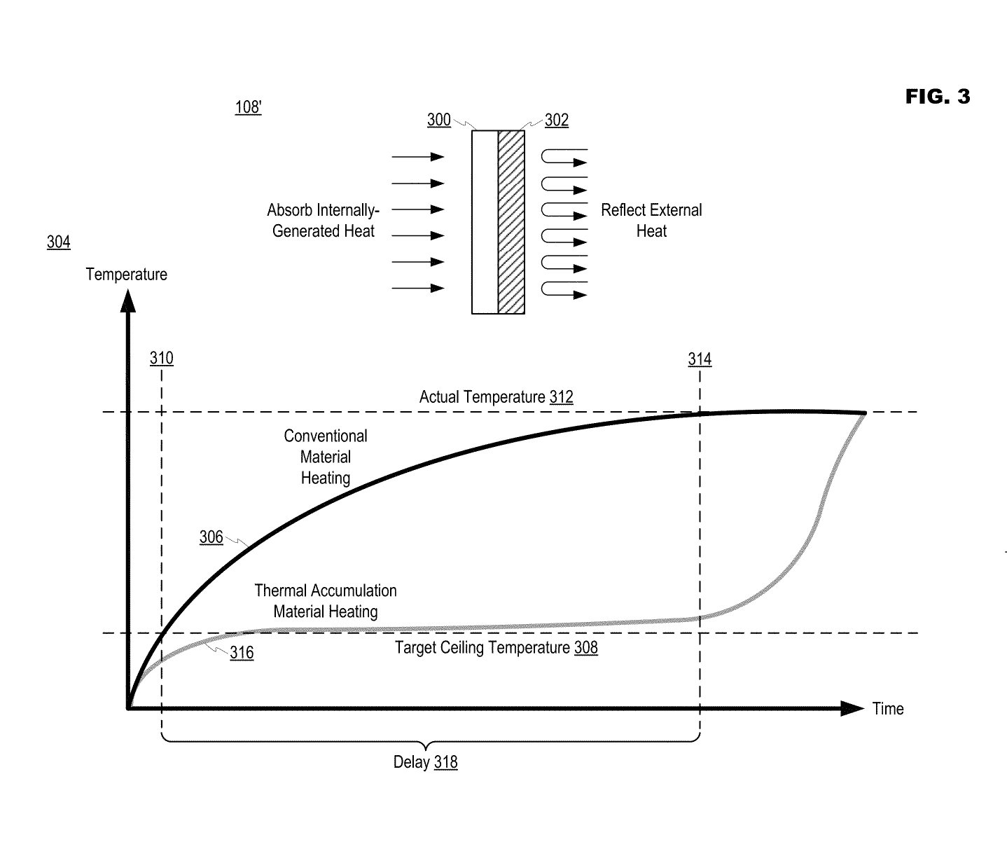

FIG. 4 illustrates example operations for protecting male reproductive health in accordance with at least one embodiment of the present disclosure. Example operations such as illustrated in FIG. 4 may be performed by a monitoring device. An internal temperature may be sensed in operation 400. A determination may then be made into operation 402 as to whether the sensed internal temperature is at or above a threshold temperature level. If in operation 402 it is determined that the internal temperature is at or above the threshold temperature level, then in operation 404 the monitoring device may cause feedback to be generated to a system user, and in optional operation 406 the monitoring device may cause an alert message may be transmitted. The alert message may be implementation-dependent (e.g., it may only be included when desired and/or needed). The alert message may be a specialized message to, for example, notify a third party regarding a potential overheating situation being experienced by the system user. Based on the alert message, the third party may notify the system user to remedy the overheating situation, may determine what caused the overheating situation so that it may be avoided in the future, etc.

Operations 408 to 410 may be optional in that they may only apply to systems including an external temperature sensor. In operation 408 an external temperature may be sensed by the external temperature sensor. A determination may then be made in operation 410 as to whether the sensed external temperature (e.g., an environmental temperature) is at or above a threshold temperature level (e.g., that may be the same threshold temperature level utilized to evaluate the internal temperature or may be a totally different threshold temperature level). If it is determined in operation 410 that the external temperature is at or above the threshold temperature level, then in operation 404 the monitoring device may cause feedback to be generated and optionally an alert message to be transmitted in operation 406. If in operation 410 it is determined that the external temperature is not at or above the threshold temperature level, then in operation 412 the monitoring device may cause a message to be transmitted, the message comprising the internal temperature and/or the external temperature (e.g., if the system is equipped for sensing external temperature). Following operation 412, internal temperature sensing may resume in operation 400.

While FIG. 4 illustrates operations according to an embodiment, it is to be understood that not all of the operations depicted in FIG. 4 are necessary for other embodiments. Indeed, it is fully contemplated herein that in other embodiments of the present disclosure, the operations depicted in FIG. 4, and/or other operations described herein, may be combined in a manner not specifically shown in any of the drawings, but still fully consistent with the present disclosure. Thus, claims directed to features and/or operations that are not exactly shown in one drawing are deemed within the scope and content of the present disclosure.

As used in this application and in the claims, a list of items joined by the term “and/or” can mean any combination of the listed items. For example, the phrase “A, B and/or C” can mean A; B; C; A and B; A and C; B and C; or A, B and C. As used in this application and in the claims, a list of items joined by the term “at least one of” can mean any combination of the listed terms. For example, the phrases “at least one of A, B or C” can mean A; B; C; A and B; A and

C; B and C; or A, B and C.

As used in any embodiment herein, the term “module” may refer to software, firmware and/or circuitry configured to perform any of the aforementioned operations. Software may be embodied as a software package, code, instructions, instruction sets and/or data recorded on non-transitory computer readable storage mediums. Firmware may be embodied as code, instructions or instruction sets and/or data that are hard-coded (e.g., nonvolatile) in memory devices. “Circuitry”, as used in any embodiment herein, may comprise, for example, singly or in any combination, hardwired circuitry, programmable circuitry such as computer processors comprising one or more individual instruction processing cores, state machine circuitry, and/or firmware that stores instructions executed by programmable circuitry. The modules may, collectively or individually, be embodied as circuitry that forms part of a larger system, for example, an integrated circuit (IC), system on-chip (SoC), desktop computers, laptop computers, tablet computers, servers, smartphones, etc.

Any of the operations described herein may be implemented in a system that includes one or more storage mediums (e.g., non-transitory storage mediums) having stored thereon, individually or in combination, instructions that when executed by one or more processors perform the methods. Here, the processor may include, for example, a server CPU, a mobile device CPU, and/or other programmable circuitry. Also, it is intended that operations described herein may be distributed across a plurality of physical devices, such as processing structures at more than one different physical location. The storage medium may include any type of tangible medium, for example, any type of disk including hard disks, floppy disks, optical disks, compact disk read-only memories (CD-ROMs), compact disk rewritables (CD-RWs), and magneto-optical disks, semiconductor devices such as read-only memories (ROMs), random access memories (RAMs) such as dynamic and static RAMs, erasable programmable read-only memories (EPROMs), electrically erasable programmable read-only memories (EEPROMs), flash memories, Solid State Disks (SSDs), embedded multimedia cards (eMMCs), secure digital input/output (SDIO) cards, magnetic or optical cards, or any type of media suitable for storing electronic instructions. Other embodiments may be implemented as software modules executed by a programmable control device.

Thus, the present application is directed to a system for protecting male reproductive health. An example system may comprise at least a temperature control portion and a monitoring device. The temperature control portion may include a thermal accumulation material to absorb heat generated by male genitalia. The monitoring device may include at least one sensor to sense an internal temperature corresponding to a surface temperature of the male genitalia, and possibly a second sensor to sense a temperature external to the temperature control portion. The monitoring device may compare the internal and/or external temperatures to threshold temperature levels, and may take action based on either/both temperatures being at or above the threshold temperature levels. For example, the monitoring device may further comprise a feedback module to generate feedback for alerting a user to a dangerous temperature condition and a communication module to send messages comprising the internal/external temperatures, alert messages, etc.

The following examples pertain to further embodiments. The following examples of the present disclosure may comprise subject material such as a device, a method, at least one machine-readable medium for storing instructions that when executed cause a machine to perform acts based on the method, means for performing acts based on the method and/or a system for protecting male reproductive health, as provided below.

According to example 1 there is provided a system for protecting male reproductive health. The system may comprise a temperature control portion including at least a material to control a temperature of a system user's genitalia and a monitoring device including at least a first sensor to sense an internal temperature, a feedback module to generate feedback to the system user and a communication module to transmit a message including at least the internal temperature.

Example 2 may include the elements of example 1, wherein the material is situated on an internal surface of the temperature control portion to direct heat away from the system user's genitalia.

Example 3 may include the elements of example 2, wherein the material is a thermal accumulation material.

Example 4 may include the elements of any of examples 2 to 3, wherein the material is a thermal conductive material.

Example 5 may include the elements of any of examples 1 to 4, wherein the temperature control portion further comprises a thermal reflective material situated on an external surface to reflect heat external to the temperature control portion.

Example 6 may include the elements of any of examples 1 to 5, wherein at least a sensing portion of the first sensor is integrated into the temperature control portion.

Example 7 may include the elements of any of examples 1 to 6, wherein the internal temperature corresponds to a sensed surface temperature of the system user's genitalia.

Example 8 may include the elements of any of examples 1 to 7, wherein the monitoring device further comprises an operations module to control operations based at least on the internal temperature.

Example 9 may include the elements of example 8, wherein the operations module is further to cause the communication module to transmit a message including at least the internal temperature.

Example 10 may include the elements of any of examples 8 to 9, wherein the operations module is further to at least one of cause the feedback module to provide feedback to the user when it is determined that the internal temperature is at or above a threshold temperature level or cause the communication module to transmit an internal temperature alert message when it is determined that the internal temperature is at or above a threshold temperature level.

Example 11 may include the elements of any of examples 8 to 10, wherein the monitoring device further comprises a second sensor to sense a temperature external to the temperature control portion.

Example 12 may include the elements of example 11, wherein the operations module is further to cause the communication module to transmit a message including at least the external temperature.

Example 13 may include the elements of any of examples 11 to 12, wherein the operations module is further to at least one of cause the feedback module to provide feedback to the user when it is determined that the external temperature data is at or above a threshold temperature level or cause the communication module to transmit an external temperature alert message when it is determined that the external temperature is at or above a threshold temperature level.

Example 14 may include the elements of any of examples 1 to 13, wherein the feedback module is to generate at least one of audible feedback, visible feedback or haptic feedback to the user.

Example 15 may include the elements of any of examples 1 to 14, wherein the system is integrated into a male undergarment or coupled to a male undergarment.

According to example 16 there is provided a device for reporting data regarding male reproductive health. The device may comprise a communication module to interact with at least one of a system for protecting male reproductive health or a remote resource and a reproductive health module to receive a message from the system for protecting male reproductive health via the communication module, the received message including at least one of an internal temperature corresponding to a surface temperature of a system user's genitalia or an external temperature corresponding to a temperature external to a temperature control portion of the system for protecting male reproductive health, generate enhanced data by correlating context data with at least one of the internal temperature or the external temperature and cause the communication module to transmit a message to the remote resource, the transmitted message including at least the enhanced data.

Example 17 may include the elements of example 16, wherein the context data comprises at least one of locational data, application data, Internet data, scheduling data or interactional data.

Example 18 may include the elements of any of examples 16 to 17, wherein the reproductive health module is further to cause the device to display information based on at least one of the internal temperature, external temperature or enhanced data.

According to example 19 there is provided a method for protecting male reproductive health. The method may comprise sensing an internal temperature corresponding to a surface temperature of a system user's genitalia, determining whether the internal temperature is at or above a first threshold temperature level and causing feedback to be generated based on a determination that the internal temperature is at or above the first threshold temperature level.

Example 20 may include the elements of example 19, and may further comprise controlling heat generated by the system user's genitalia.

Example 21 may include the elements of any of examples 19 to 20, and may further comprise sensing an external temperature corresponding to a temperature sensed external to a temperature control portion of a system for protecting male reproductive health, determining whether the external temperature is at or above a second threshold temperature level and causing a feedback to be generated based on a determination that the external temperature is at or above the second threshold temperature level.

Example 22 may include the elements of example 21, and may further comprise reflecting heat external to the temperature control portion.

Example 23 may include the elements of any of examples 21 to 22, and may further comprise causing a message to be transmitted, the message comprising at least one of the internal temperature or the external temperature.

Example 24 may include the elements of any of examples 21 to 23, and may further comprise causing an alert message to be transmitted if is determined that at least one of the internal temperature is at or above the first threshold temperature level or the external temperature is at or above the second threshold temperature level.

According to example 25 there is provided a method for reporting data regarding male reproductive health. The method may comprise receiving a message from a system for protecting male reproductive health, the received message including at least one of an internal temperature corresponding to a surface temperature of a system user's genitalia or an external temperature corresponding to a temperature external to a temperature control portion of the system for protecting male reproductive health, generating enhanced data by correlating context data with at least one of the internal temperature or the external temperature and causing a message to be transmitted to a remote resource, the transmitted message including at least the enhanced data.

Example 26 may include the elements of example 25, wherein the context data comprises at least one of locational data, application data, Internet data, scheduling data or interactional data.

Example 27 may include the elements of any of examples 25 to 26, and may further comprise causing the device to display information based on at least one of the internal temperature, the external temperature or the enhanced data.

According to example 28 there is provided a system including at least one device, the system being arranged to perform the method of any of the above examples 19 to 27.

According to example 29 there is provided a chipset arranged to perform the method of any of the above examples 19 to 27.

According to example 30 there is provided at least one machine readable medium comprising a plurality of instructions that, in response to be being executed on a computing device, cause the computing device to carry out the method according to any of the above examples 19 to 27.

According to example 31 there is provided at least one device configured for protecting male reproductive health, the at least one device being arranged to perform the method of any of the above examples 19 to 27.

According to example 32 there is provided a system for protecting male reproductive health. The system may comprise means for sensing an internal temperature corresponding to a surface temperature of a system user's genitalia, means for determining whether the internal temperature is at or above a first threshold temperature level and means for causing feedback to be generated based on a determination that the internal temperature is at or above the first threshold temperature level.

Example 33 may include the elements of example 32, and may further comprise means for sensing an external temperature corresponding to a temperature sensed external to a temperature control portion of a system for protecting male reproductive health, means for determining whether the external temperature is at or above a second threshold temperature level and means for causing feedback to be generated based on a determination that the external temperature is at or above the second threshold temperature level.

Example 34 may include the elements of example 33, and may further comprise means for causing a message to be transmitted, the message comprising at least one of the internal temperature or the external temperature.

Example 35 may include the elements of any of examples 33 to 34, and may further comprise means for causing an alert message to be transmitted if is determined that at least one of the internal temperature is at or above the first threshold temperature level or the external temperature is at or above the second threshold temperature level.

According to example 36 there is provided a system for reporting data regarding male reproductive health. The system may comprise means for receiving a message from a system for protecting male reproductive health, the received message including at least one of an internal temperature corresponding to a surface temperature of a system user's genitalia or an external temperature corresponding to a temperature external to a temperature control portion of the system for protecting male reproductive health, means for generating enhanced data by correlating context data with at least one of the internal temperature or the external temperature; and means for causing a message to be transmitted to a remote resource, the transmitted message including at least the enhanced data.

Example 37 may include the elements of example 36, wherein the context data comprises at least one of locational data, application data, Internet data, scheduling data or interactional data.

Example 38 may include the elements of any of examples 36 to 37, and may further comprise causing the device to display information based on at least one of the internal temperature, the external temperature or the enhanced data.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents.

What is claimed:

1. A system for protecting male reproductive health, comprising:
a temperature control portion including at least a material to control a temperature of a system user's genitalia; and
a monitoring device including at least:
a first sensor to sense an internal temperature;
a feedback module to generate feedback to the system user; and
a communication module to transmit a message including at least the internal temperature.

2. The system of claim 1, wherein the material is situated on an internal surface of the temperature control portion to direct heat away from the system user's genitalia.

3. The system of claim 2, wherein the material is a thermal accumulation material.

4. The system of claim 1, wherein the temperature control portion further comprises a thermal reflective material situated on an external surface to reflect heat external to the temperature control portion.

5. The system of claim 1, wherein at least a sensing portion of the first sensor is integrated into the temperature control portion.

6. The system of claim 1, wherein the internal temperature corresponds to a sensed surface temperature of the system user's genitalia.

7. The system of claim 1, wherein the monitoring device further comprises an operations module to control operations based at least on the internal temperature.

8. The system of claim 7, wherein the operations module is further to cause the communication module to transmit a message including at least the internal temperature.

9. The system of claim 7, wherein the operations module is further to at least one of:
cause the feedback module to provide feedback to the user when it is determined that the internal temperature is at or above a threshold temperature level; or
cause the communication module to transmit an internal temperature alert message when it is determined that the internal temperature is at or above a threshold temperature level.

10. The system of claim 7, wherein the monitoring device further comprises a second sensor to sense a temperature external to the temperature control portion.

11. The system of claim 10, wherein the operations module is further to cause the communication module to transmit a message including at least the external temperature.

12. The system of claim 10, wherein the operations module is further to at least one of:
cause the feedback module to provide feedback to the user when it is determined that the external temperature data is at or above a threshold temperature level; or
cause the communication module to transmit an external temperature alert message when it is determined that the external temperature is at or above a threshold temperature level.

13. The system of claim 1, wherein the feedback module is to generate at least one of audible feedback, visible feedback or haptic feedback to the user.

14. A device for reporting data regarding male reproductive health, comprising:
a communication module to interact with at least one of a system for protecting male reproductive health or a remote resource; and
a reproductive health module to:
receive a message from the system for protecting male reproductive health via the communication module, the received message including at least one of an internal temperature corresponding to a surface temperature of a system user's genitalia or an external temperature corresponding to a temperature external to a temperature control portion of the system for protecting male reproductive health;
generate enhanced data by correlating context data with at least one of the internal temperature or the external temperature; and
cause the communication module to transmit a message to the remote resource, the transmitted message including at least the enhanced data.

15. The device of claim 13, wherein the context data comprises at least one of locational data, application data, Internet data, scheduling data or interactional data.

16. A method for protecting male reproductive health, comprising:
sensing an internal temperature corresponding to a surface temperature of a system user's genitalia;
determining whether the internal temperature is at or above a first threshold temperature level; and
causing feedback to be generated based on a determination that the internal temperature is at or above the first threshold temperature level.

17. The method of claim 16, further comprising:
controlling heat generated by the system user's genitalia.

18. The method of claim 16, further comprising:
sensing an external temperature corresponding to a temperature sensed external to a temperature control portion of a system for protecting male reproductive health;
determining whether the external temperature is at or above a second threshold temperature level; and
causing a feedback to be generated based on a determination that the external temperature is at or above the second threshold temperature level.

19. The method of claim 18, further comprising:
reflecting heat external to the temperature control portion.

20. The method of claim 18, further comprising:
causing a message to be transmitted, the message comprising at least one of the internal temperature or the external temperature.

21. The method of claim 18, further comprising:
causing an alert message to be transmitted if is determined that at least one of the internal temperature is at or above the first threshold temperature level or the external temperature is at or above the second threshold temperature level.

22. At least one machine-readable storage medium having stored thereon, individually or in combination, instructions for protecting male reproductive health that, when executed by one or more processors, cause the one or more processors to:
sense an internal temperature corresponding to a surface temperature of a system user's genitalia;
determine whether the internal temperature is at or above a first threshold temperature level; and
cause feedback to be generated based on a determination that the internal temperature is at or above the first threshold temperature level.

23. The medium of claim 22, further comprising instructions that, when executed by one or more processors, cause the one or more processors to:
sense an external temperature corresponding to a temperature sensed external to a temperature control portion of a system for protecting male reproductive health;
determine whether the external temperature is at or above a second threshold temperature level; and
cause feedback to be generated based on a determination that the external temperature is at or above the second threshold temperature level.

24. The medium of claim 23, further comprising instructions that, when executed by one or more processors, cause the one or more processors to:
cause a message to be transmitted, the message comprising at least one of the internal temperature or the external temperature.

25. The medium of claim 23, further comprising instructions that, when executed by one or more processors, cause the one or more processors to:
cause an alert message to be transmitted if is determined that at least one of the internal temperature is at or above the first threshold temperature level or the external temperature is at or above the second threshold temperature level.
